(19) United States
(12) Patent Application Publication
(10) Pub. No.: US 2021/0128225 A1
(43) Pub. Date: May 6, 2021

(54) CONTROLLER, TREATMENT SYSTEM, AND METHOD FOR ACTUATING CONTROLLER

(71) Applicant: OLYMPUS CORPORATION, Tokyo (JP)

(72) Inventor: Shun IZUMI, Hachioji-shi (JP)

(73) Assignee: OLYMPUS CORPORATION, Tokyo (JP)

(21) Appl. No.: 17/143,246

(22) Filed: Jan. 7, 2021

Related U.S. Application Data
(63) Continuation of application No. PCT/JP2018/026045, filed on Jul. 10, 2018.

(51) Int. Cl. A61B 18/14; G16H 20/30; G16H 40/63

(52) U.S. Cl. CPC A61B 18/1445; A61B 2018/00589; G16H 40/63; G16H 20/30

(57) ABSTRACT

A controller is used together with an energy treatment instrument including a first grasping piece, a second grasping piece, a reference electrode provided in the first grasping piece, a first counter electrode provided in the second grasping piece, and a second counter electrode provided in the second grasping piece. The controller includes a processor that acquires a first parameter relating to a thickness of a first portion of the treatment target, which is grasped between the reference electrode and the first counter electrode, and a second parameter relating to a thickness of a second portion of the treatment target, which is grasped between the reference electrode and the second counter electrode. The processor controls an amount of electric energy applied to one of the first portion or the second portion of the treatment target to be larger than another one of the first portion or the second portion.

### CROSS-REFERENCE TO RELATED APPLICATIONS

This is a Continuation Application of PCT Application No. PCT/JP2018/026045, filed Jul. 10, 2018, the entire contents of which are incorporated herein by reference.

### BACKGROUND

In an energy treatment instrument, an electrode is provided in each of a pair of grasping pieces. When a high-frequency power is supplied to each of the electrodes, a high-frequency current flows between the electrodes through a living tissue grasped between the pair of grasping pieces. The living tissue is coagulated (sealed) by the flow of the high-frequency current.

In a treatment using the energy treatment instrument, the thickness of the living tissue grasped between the pair of grasping pieces may not be uniform. In this case, treatment performance of the energy treatment instrument may be affected.

### SUMMARY

The present embodiment(s) relates to a controller used together with an energy treatment instrument, a treatment system, and a method for actuating the controller.

According to an exemplary embodiment, a controller used together with an energy treatment instrument, the energy treatment including a first grasping piece, a second grasping piece configured to grasp a treatment target by opening and closing relative to the first grasping piece, a reference electrode provided in the first grasping piece, a first counter electrode provided in the second grasping piece, and a second counter electrode provided in the second grasping piece, the controller comprising: a processor configured to: acquire (i) a first parameter relating to a thickness of a first portion of the treatment target, the first portion grasped between the reference electrode and the first counter electrode, and (ii) a second parameter relating to a thickness of a second portion of the treatment target, the second portion grasped between the reference electrode and the second counter electrode; calculate a difference value between the first parameter and the second parameter; and control an amount of electric energy applied to one of the first portion or the second portion, which has a greater thickness, of the treatment target, such that the amount of electric energy applied is larger than an amount of electric energy applied to another one of the first portion or the second portion, which has a lesser thickness, of the treatment target when the calculated difference value is greater than a predetermined value.

According to an exemplary embodiment, a treatment system includes: an energy treatment instrument including: a first grasping piece, a second grasping piece configured to grasp a treatment target by opening and closing relative to the first grasping piece, a reference electrode provided in the first grasping piece, a first counter electrode provided in the second grasping piece, and a second counter electrode provided in the second grasping piece; and a controller including a processor configured to: acquire (i) a first parameter relating to a thickness of a first portion of the treatment target, the first portion being grasped between the reference electrode and the first counter electrode, and (ii) a second parameter relating to a thickness of a second portion of the treatment target, the second portion being grasped between the reference electrode and the second counter electrode, calculate a difference value between the first parameter and the second parameter, and control an amount of electric energy applied to one of the first portion or the second portion, which has a greater thickness, of the treatment target, such that the amount of electric energy applied is larger than an amount of electric energy applied to another one of the first portion or the second portion, which has a lesser thickness, of the treatment target when the calculated difference value is greater than a predetermined value.

According to an exemplary embodiment, a method for actuating the controller includes: acquiring the first parameter relating to the thickness of the first portion of the treatment target, the first portion being grasped between the reference electrode and the first counter electrode, and the second parameter relating to the thickness of the second portion of the treatment target, the second portion being grasped between the reference electrode and the second counter electrode; calculating the difference value between the first parameter and the second parameter; and controlling the amount of the electric energy applied to the one of the first portion or the second portion, which has the greater thickness, of the treatment target, such that the amount of electric energy applied is larger than the amount of the electric energy applied to the other one of the first portion or the second portion, which has the lesser thickness, of the treatment target when the calculated difference value is greater than the predetermined value.

### BRIEF DESCRIPTION OF THE DRAWINGS

FIG. 1 is a diagram schematically showing a treatment system according to a first embodiment.

FIG. 2 is a block diagram schematically showing an electrical connection state in the treatment system according to the first embodiment.

FIG. 3 is a diagram schematically showing an end effector of the energy treatment instrument according to the first embodiment, in a cross section approximately perpendicular to a longitudinal axis.

FIG. 4 is a schematic diagram showing a state in which a treatment target is grasped between grasping pieces of the energy treatment instrument according to the first embodiment, in a cross section approximately perpendicular to the longitudinal axis.

FIG. 5 is a flowchart showing processing performed by a processor of a controller according to the first embodiment in a coagulation treatment of the treatment target.

FIG. 6 is a flowchart showing processing performed by a processor of a controller according to a second embodiment in a coagulation treatment of a treatment target.

FIG. 7 is a flowchart showing processing performed by a processor of a controller according to a third embodiment in a coagulation treatment of a treatment target.

FIG. 8 is a block diagram schematically showing an electrical connection state in a treatment system according to a fourth embodiment.

FIG. 9 is a flowchart showing processing performed by a processor of a controller according to the fourth embodiment in a coagulation treatment of a treatment target.

FIG. 10 is a flowchart showing processing performed by a processor of a controller according to a first modification of the fourth embodiment in a coagulation treatment of a treatment target.

FIG. 11 is a schematic diagram showing a state in which a treatment target is grasped between grasping pieces of an energy treatment instrument according to a fifth embodiment, in a cross section approximately perpendicular to a width direction.

FIG. 12 is a block diagram schematically showing an electrical connection state in a treatment system according to the fifth embodiment.

FIG. 13 is a block diagram schematically showing an electrical connection state in a treatment system according to a sixth embodiment.

### DETAILED DESCRIPTION

### First Embodiment

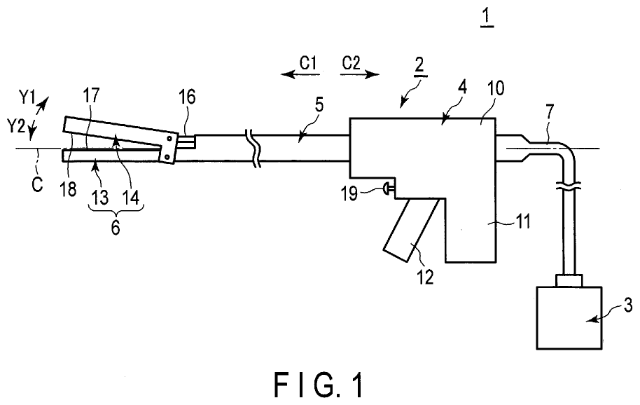

FIG. 1 is a diagram showing a treatment system 1 which is a treatment system of the present embodiment. As shown in FIG. 1, the treatment system 1 includes a treatment instrument 2, which is an energy treatment instrument, and a controller (power source device) 3. The treatment instrument 2 includes a housing 4 and a tubular shaft 5 coupled to the housing 4. The housing 4 is holdable. One end of a cable 7 is connected to the housing 4. The other end of the cable 7 is detachably connected to the controller 3.

The shaft 5 defines a longitudinal axis C. Here, a direction along the longitudinal axis C is referred to as a longitudinal direction. One side of the longitudinal direction is referred to as a distal side (an arrow Cl side in FIG. 1), and a side opposite to the distal side is referred to as a proximal side (an arrow C2 side in FIG. 1). The shaft 5 is coupled to the distal side of the housing 4, and extends along the longitudinal axis C from the proximal side to the distal side.

An end effector 6 is provided in a distal end of the shaft 5. The end effector 6 includes a first grasping piece (first grasping member) 13 and a second grasping piece (second grasping member) 14. The first grasping piece 13 and the second grasping piece 14 can be opened and closed relative to each other. The first grasping piece 13 is supported by the shaft 5. The second grasping piece 14 is rotatably attached to the distal end of the shaft 5, and is rotatable relative to the first grasping piece 13. Both of the first grasping piece 13 and the second grasping piece 14 may be attached rotatably relative to the shaft 5.

The first grasping piece 13 includes a treatment surface (opposing face) 17 that applies treatment energy to a treatment target. The treatment surface 17 faces the second grasping piece 14. The second grasping piece 14 includes a treatment surface (opposing face) 18 that applies treatment energy to a treatment target. The treatment surface 18 faces the treatment surface 17 of the first grasping piece 13.

The opening and closing directions of the end effector intersect with the longitudinal axis C and are perpendicular or approximately perpendicular to the longitudinal axis C. Of the opening and closing directions of the end effector 6, a side on which the second grasping piece 14 opens relative to the first grasping piece 13 is defined as an opening direction (arrow Y1) of the second grasping piece 14, and a side on which the second grasping piece 14 closes relative to the first grasping piece 13 is defined as a closing direction (arrow Y2) of the second grasping piece 14. Also, a direction intersecting with (perpendicular or approximately perpendicular to) the longitudinal axis C and intersecting with (perpendicular or approximately perpendicular to) the opening and closing directions of the end effector 6 is defined as a width direction of the end effector 6.

The housing 4 includes a housing main body 10 and a grip (fixed handle) 11. The housing main body 10 extends along the longitudinal axis C. The grip 11 extends from the housing main body 10 toward a side away from the longitudinal axis C. The shaft 5 is coupled to the housing main body 10 from the distal side.

A movable handle 12 is rotatably attached to the housing main body 10. The movable handle 12 is located near the grip 11 with respect to the longitudinal axis C, and is, in the present embodiment, located on the distal side with respect to the grip 11. When the movable handle 12 rotates relative to the housing main body 10, the movable handle 12 opens or closes relative to the grip 11. When the movable handle 12 opens or closes relative to the grip 11, an operation to cause the end effector 6 to open or close in the manner described above is input at the movable handle 12. That is, the movable handle 12 is an open/close operation input section.

The movable handle 12 and the second grasping piece 14 are coupled to each other via a movable member 16. The movable member 16 extends along the longitudinal axis C inside the shaft 5. When the movable handle 12 opens or closes with respect to the grip 11, the movable member 16 moves along the longitudinal axis C relative to the shaft 5 and the housing 4, and the second grasping piece 14 rotates relative to the shaft 5. Thus, the grasping pieces 13 and 14 open or close relative to each other. A treatment target is grasped between the grasping pieces 13 and 14 by the grasping pieces 13 and 14 closing relative to each other in a state in which the treatment target is disposed between the grasping pieces 13 and 14.

In another example, an operation member, such as a rotary knob, is attached to the housing main body 10. In this case, when the operation member is rotated about the longitudinal axis C relative to the housing 4, the shaft 5 and the end effector 6 rotate together with the operation member about the longitudinal axis C relative to the housing 4.

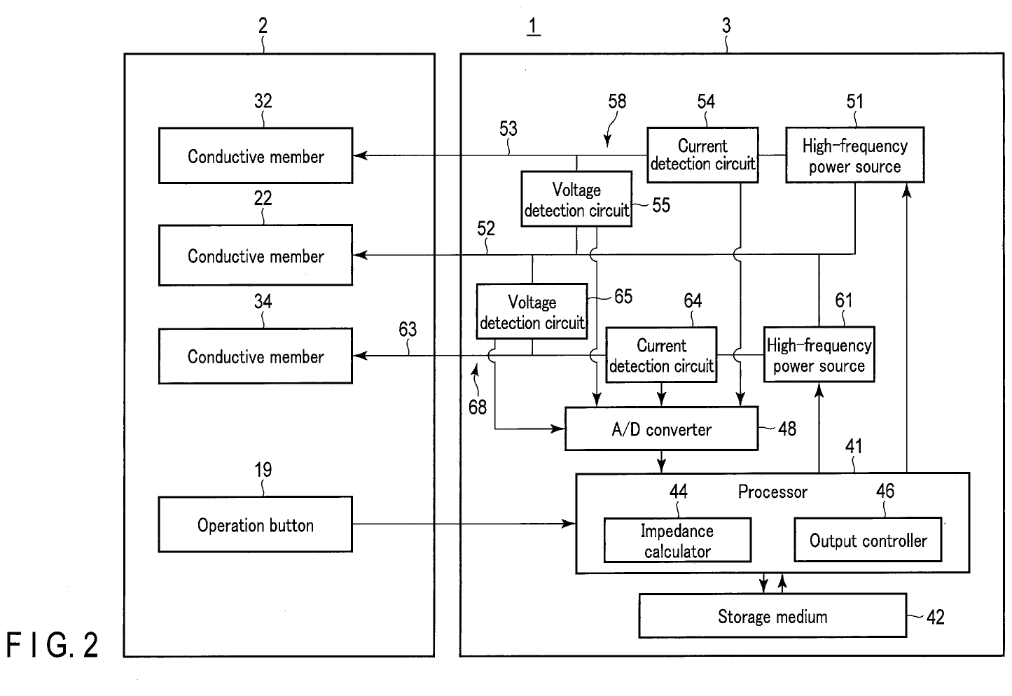

FIG. 2 is a block diagram showing a control configuration in the treatment system 1. As shown in FIG. 2, the controller 3 includes a processor (controller) 41 that controls the entire treatment system 1, and a storage medium (memory) 42. The processor 41 is formed of an integrated circuit including, for example, a central processing unit (CPU), an application specific integrated circuit (ASIC), or a field programmable gate array (FPGA).

The processor 41 may be formed of one integrated circuit or a plurality of integrated circuits. The processing in the processor 41 is performed in accordance with a program stored in the processor 41 or the storage medium 42. Further, a processing program used by the processor 41, parameters, a table, etc. used in computations by the processor 41 are stored in the storage medium 42. In one example, a processor is provided in the treatment instrument 2, and at least a part of processing to be described later is performed by the processor provided in the treatment instrument 2. In this case, the processor provided in the treatment instrument 2 also constitutes a controller that controls supply of electric energy for actuating the treatment instrument 2 to the treatment instrument 2. In this case, the storage medium 42 may be provided in the treatment instrument 2.

The controller 3 includes high-frequency power sources 51 and 61. Each of the high-frequency power sources 51 and 61 includes a waveform generator, a conversion circuit, a transformer, etc., and converts power from a battery power source, an outlet power source, etc. into high-frequency power. Furthermore, at least a part of each of the first grasping piece 13 and the second grasping piece 14 is formed of an electrically conductive material such as metal. Each of the high-frequency power sources 51 and 61 is electrically connected to the portion formed of an electrically conductive material in each of the first grasping piece 13 and the second grasping piece 14 via an electric path provided through the inside of the cable 7, the inside of the housing 4, and the inside of the shaft 5. Each of the high-frequency power sources 51 and 61 outputs the converted high-frequency power through the above-described electric path, and supplies the high-frequency power to the first grasping piece 13 and the second grasping piece 14 as electric energy. In a state in which a treatment target is grasped between the first grasping piece 13 and the second grasping piece 14, the high-frequency power is supplied to the first grasping piece 13 and the second grasping piece 14, whereby the high-frequency current flows between the first grasping piece 13 and the second grasping piece 14 via the treatment target. As a result, the high-frequency current is applied to the treatment target as treatment energy.

An operation button 19 is provided in the housing main body 10. The operation button 19 is an energy operation input section. When an operation is input by the operation button 19 in a state in which the treatment target is grasped between the grasping pieces 13 and 14, electric energy is supplied from the high-frequency power sources 51 and 61 to the treatment instrument 2. Then, a high-frequency current is applied to the grasped treatment target as treatment energy. In one example, instead of the operation button 19 or in addition to the operation button 19, a foot switch electrically connected to the controller is provided as an energy operation input section separately from the treatment instrument 2.

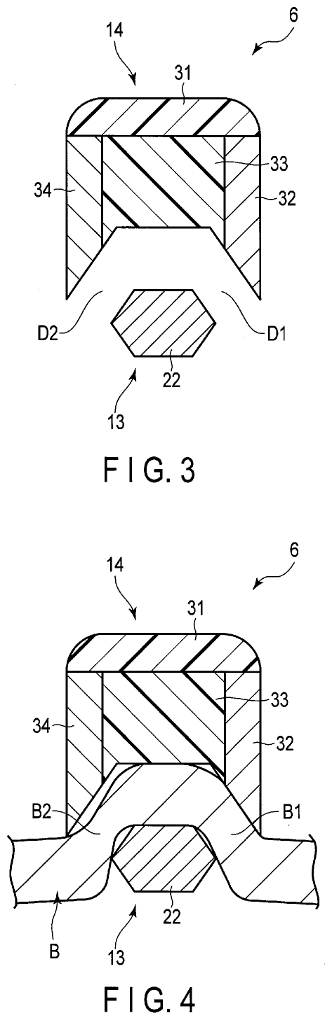

FIG. 3 is a diagram showing the end effector 6. FIG. 3 shows the end effector 6 in a cross section perpendicular or approximately perpendicular to the longitudinal axis C. In the present embodiment, the first grasping piece 13 is fixed to the distal end of the shaft 5. As shown in FIG. 3, the first grasping piece 13 includes a conductive member 22. The conductive member 22 extends along the longitudinal axis C. The conductive member 22 has conductivity. The treatment surface 17 is formed by the conductive member 22. The second grasping piece 14 includes a support 31.

The support 31 extends along the longitudinal axis C in a state in which the grasping piece 14 is closed with respect to the grasping piece 13. That is, in a state in which the grasping piece 14 is closed with respect to the grasping piece 13, an extending direction of the support 31 is parallel or approximately parallel to the longitudinal axis C. A proximal end of the support 31 is attached to the distal end of the shaft 5. The support 31 is formed of, for example, a resin material having electrical insulation properties, etc. In the present embodiment, the support 31 is rotatable relative to the shaft 5.

Conductive members 32 and 34 and an abutment member (pad member) 33 are attached to the support 31 from the grasping piece 13 side. Each of the conductive members 32 and 34 and the abutment member 33 faces the conductive member 22 and the treatment surface 17 of the first grasping piece 13. Each of the conductive members 32 and 34 and the abutment member 33 forms a part of the treatment surface 18. Each of the conductive members 32 and 34 and the abutment member 33 extends over a range from the distal end to the proximal end of the support 31 in an extending direction of the grasping piece 14.

Each of the conductive members 32 and 34 has conductivity. The conductive members 32 and 34 are made of, for example, metal such as stainless steel. The conductive members 32 and 34 are separated from each other in the width direction. The abutment member 33 is disposed between the conductive member 32 and the conductive member 34. The abutment member 33 is formed of a material having electrical insulation properties. By the abutment member 33 provided between the conductive member 32 and the conductive member 34, the conductive member 32 and the conductive member 34 are electrically insulated from each other.

Here, between the treatment surfaces 17 and 18, a space formed between the conductive members 22 and 32 is referred to as a first region D1, and a space formed between the conductive members 22 and 34 is referred to as a second region D2. In the present embodiment, the positions of the first region D1 and the second region D2 in the width direction are different from each other. In the treatment system 1, each of the high-frequency power sources 51 and 61 is electrically connected to the conductive member 22 via an electric path 52 formed of electric wiring, etc. Further, the high-frequency power source (first power source) 51 is electrically connected to the conductive member 32 via an electric path 53 formed of electric wiring, etc. Then, the high-frequency power source (second power source) 61 is electrically connected to the conductive member 34 via an electric path 63 formed of electric wiring, etc. Each of the electric paths 52, 53, and 63 extends through the inside of the cable 7, the inside of the housing 4, and the inside of the shaft 5.

When the high-frequency power is output from the high-frequency power source 51, a voltage (potential difference) is applied between the conductive members 22 and 32. In addition, when the high-frequency power is output from the high-frequency power source 61, a voltage (potential difference) is applied between the conductive members 22 and 34. Thus, when the high-frequency power is supplied from the high-frequency power sources 51 and 61, the conductive member (reference electrode) 22, the conductive member (first counter electrode) 32, and the conductive member (second counter electrode) 34 function as electrodes different from each other.

The processor 41 causes the high-frequency power sources 51 and 61 to output the high-frequency power as the electric energy based on the operation at the operation button 19. The high-frequency power output from the high-frequency power source 51 is supplied to the conductive member 22 of the grasping piece 13 via the electric path 52 and is supplied to the conductive member 32 of the grasping piece 14 via the electric path 53. Thus, the conductive member 22 and the conductive member 32 function as electrodes having potentials different from each other. When the conductive member 22 and the conductive member 32 function as electrodes in a state in which the treatment target is grasped between the grasping pieces 13 and 14, an electric circuit 58 including the high-frequency power source 51, electric path 52, conductive member 22, conductive member 32, and electric path 53 is formed, and the high-frequency current flows through the electric circuit 58. Accordingly, the high-frequency current flows between the conductive member 22 and the conductive member 32 through the treatment target grasped in the first region D1. That is, the high-frequency current is applied as the treatment energy to the treatment target grasped in the first region D1.

The high-frequency power output from the high-frequency power source 61 is supplied to the conductive member 22 of the grasping piece 13 via the electric path 52, and is supplied to the conductive member 34 of the grasping piece 14 via the electric path 63. Thus, the conductive member 22 and the conductive member 34 function as electrodes having potentials different from each other. When the conductive member 22 and the conductive member 34 function as electrodes in a state in which the treatment target is grasped between the grasping pieces 13 and 14, an electric circuit 68 including the high-frequency power source 61, electric path 52, conductive member 22, conductive member 34, and electric path 63 is formed, and the high-frequency current flows through the electric circuit 68. Accordingly, the high-frequency current flows between the conductive member 22 and the conductive member through the treatment target grasped in the second region D2. That is, the high-frequency current is applied as the treatment energy to the treatment target grasped in the second region D2.

As described above, in the present embodiment, the high-frequency power is output from both of the high-frequency power sources 51 and 61 based on the operation at the operation button 19, and the high-frequency current is simultaneously applied to the grasped treatment target in the first region D1 and the second region D2.

A current detection circuit 54 and a voltage detection circuit 55 are provided in the electric circuit 58. The current detection circuit 54 detects a current value I1 of an output current from the high-frequency power source 51 to the electric circuit 58. The voltage detection circuit 55 detects a voltage value V1 of an output voltage from the high-frequency power source 51 to the electric circuit 58. The voltage value V1 of the output voltage is the same or approximately the same as the potential difference between the conductive member 22 (reference electrode) and the conductive member 32 (first counter electrode).

A current detection circuit 64 and a voltage detection circuit 65 are provided in the electric circuit 68. The current detection circuit 64 detects a current value 12 of an output current from the high-frequency power source 61 to the electric circuit 68. The voltage detection circuit 65 detects a voltage value V2 of an output voltage from the high-frequency power source 61 to the electric circuit 68. The voltage value V2 is the same or approximately the same as the potential difference between the conductive member 22 (reference electrode) and the conductive member 34 (second counter electrode).

An A/D converter 48 is provided in the controller 3. Analog signals relating to the current values I1 and I2 detected in the current detection circuits 54 and 64, and analog signals relating to the voltage values V1 and V2 detected in the voltage detection circuits 55 and 65 are transmitted to the A/D converter 48. The A/D converter 48 converts the analog signals relating to the current values I1 and I2 and the analog signals relating to the voltage values V1 and V2 into digital signals, and transmits the converted digital signals to the processor 41.

The processor 41 includes an impedance calculator 44 and an output controller 46. The impedance calculator 44 and the output controller 46 function as a part of the processor 41, and perform a part of processing performed by the processor 41.

The processor 41 acquires the current values I1 and I2 and the voltage values V1 and V2. The impedance calculator 44 of the processor 41 calculates an impedance value (first impedance value) Z1 in the electric circuit (first electric circuit) 58 based on the current value I1 and the voltage value V1. The impedance calculator 44 of the processor 41 calculates an impedance value (second impedance value) Z2 in the electric circuit (second electric circuit) 68 based on the current value 12 and the voltage value V2. Equations, tables, etc. used to calculate the impedance values Z1 and Z2 are stored in, for example, the storage medium 42.

The processor 41 acquires the calculated impedance values Z1 and Z2 as parameters relating to the thickness of the grasped treatment target. At this time, the processor 41 acquires the impedance value Z1 as a parameter (first parameter) relating to the thickness of a portion of the treatment target that is grasped between the reference electrode (22) and the first counter electrode (32). In addition, the processor 41 acquires the impedance value Z2 as a parameter (second parameter) relating to the thickness of a portion of the treatment target that is grasped between the reference electrode (22) and the second counter electrode (34).

The processor 41 detects whether or not there is any operation input at the energy operation input section such as the operation button 19. The output controller 46 of the processor 41 controls the output of the electric energy from the high-frequency power sources 51 and 61 based on a detection result of the operation input at the operation button 19 and the parameters relating to the thickness of the treatment target.

Next, functions and advantageous effects of the treatment instrument 2, controller 3, and treatment system 1 of the present embodiment are described. The treatment system 1 of the present embodiment is, for example, used for a treatment of grasping a living tissue such as a blood vessel as a treatment target and applying treatment energy such as a high-frequency current to the grasped living tissue so as to coagulate the living tissue.

In the treatment using the treatment system 1, first, the end effector 6 is inserted into a body cavity such as an abdominal cavity. Then, a treatment target such as a blood vessel is disposed between the pair of grasping pieces 13 and 14, and the end effector 6 is closed. Thus, the treatment target is grasped between the grasping pieces and 14. In a state in which the treatment target is grasped between the grasping pieces 13 and 14, an operation input for supplying electric energy from the controller 3 to the treatment instrument 2 is performed, whereby a high-frequency current is applied to the grasped treatment target as described above. Thus, the treatment target is coagulated.

FIG. 4 is a diagram showing a state in which a blood vessel B is grasped as a treatment target between the pair of grasping pieces 13 and 14. The blood vessel B is disposed along the width direction of the end effector 6 between the grasping pieces 13 and 14. Here, in the blood vessel B, a portion grasped between the conductive members and 32 is referred to as a first portion B1, and a portion grasped between the conductive members 22 and 34 is referred to as a second portion B2. The first portion B1 is a portion disposed in the first region D1, and the second portion B2 is a portion disposed in the second region D2. That is, the first portion B1 is a portion disposed between the reference electrode (22) and the first counter electrode (32), and the second portion B2 is a portion disposed between the reference electrode (22) and the second counter electrode (34). The positions of the first portion B1 and the second portion B2 in the width direction are different from each other.

When the blood vessel B is grasped between the grasping pieces 13 and 14, the thickness of the grasped blood vessel B may not be uniform. In this case, the thickness of the grasped blood vessel B varies depending on the position. For example, in a case where thickness T1 of the first portion B1 and thickness T2 of the second portion B2 are different from each other, the first portion B1 and the second portion B2 are different in contact area with the electrodes. Thus, the impedance value Z1 of the electric circuit 58 that passes the high-frequency current through the first portion B1 and the impedance value Z2 of the electric circuit 68 that passes the high-frequency current through the second portion B2 are different from each other.

For example, as shown in FIG. 4, the thickness T1 of the first portion B1 may be larger (thicker) than the thickness T2 of the second portion B2. In this case, the thickness of the grasped blood vessel B is different in the width direction. An area where the first portion B1 of the blood vessel B contacts the electrodes 22 and 32 in the first region D1 is larger than an area where the second portion B2 of the blood vessel B contacts the electrodes 22 and 34 in the second region D2. For this reason, the impedance value Z1 of the electric circuit 58 that passes the high-frequency current through the first portion B1 is smaller than the impedance value Z2 of the electric circuit 68 that passes the high-frequency current through the second portion B2.

Further, for example, in a case where the thickness T1 of the first portion B1 is smaller (thinner) than the thickness T2 of the second portion B2 in the blood vessel B, the impedance value Z1 of the electric circuit 58 that passes the high-frequency current through the first portion B1 is greater than the impedance value Z2 of the electric circuit 68 that passes the high-frequency current through the second portion B2.

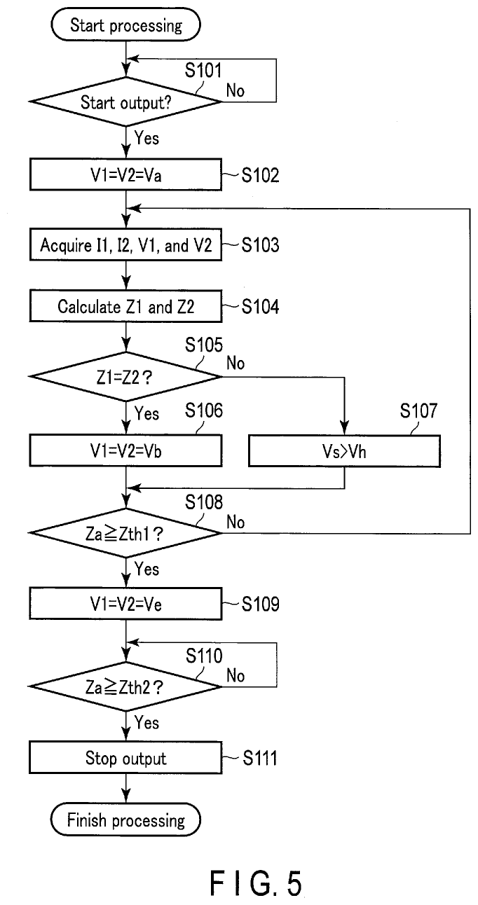

FIG. 5 is a flowchart showing processing performed by the processor 41 in the treatment of applying the high-frequency current to the blood vessel B. As shown in FIG. 5, in the coagulation treatment of the blood vessel B, the processor 41 first determines whether or not an operation for outputting electric energy from the controller 3 to the treatment instrument 2 is input at the operation button 19 (S101).

If an electric signal indicating that an output operation is input at the operation button 19 is not detected (S101-No), the processor 41 continues a standby state and maintains the standby state until an electric signal indicating that an output operation is input is detected.

When the electric signal indicating that the output operation is input at the operation button 19 is detected (S101-Yes), the processor 41 starts output control in a first output mode. At this time, the processor 41 starts the output of the electric energy from the high-frequency power sources 51 and 61 of the controller 3 to the conductive members 22, 32, and 34 of the treatment instrument 2. Thus, the application of the high-frequency current to the grasped blood vessel B is started as described above. In the present embodiment, the processor 41 performs control to set each of the voltage values V1 and V2 to a predetermined value (set value) by controlling the output from the high-frequency power sources 51 and 61 to the treatment instrument 2. In the first output mode, the processor 41 first performs control to maintain, with time, the voltage values V1 and V2 at a set value Va (S102). Thus, the voltage values V1 and V2 become the same value. The set value Va is an output value for detecting the thickness of the grasped tissue. The set value Va is, for example, smaller than a voltage value during the coagulation treatment. The set value Va is stored in, for example, the storage medium 42.

In the first output mode, the processor 41 then acquires the current values I1 and I2 detected by the current detection circuits 54 and 64 and the voltage values V1 and V2 detected by the voltage detection circuits 55 and 65 (S103).

In the first output mode, the processor 41 then calculates the impedance values Z1 and Z2 for the electric circuits 58 and 68, respectively, based on the voltage values V1 and V2 and the current values I1 and I2 (S104). In addition, the processor 41 calculates a reference value Za of impedance. The reference value Za is, for example, an average value of the impedance values Z1 and Z2.

In the first output mode, the processor 41 then determines whether the impedance values Z1 and Z2 are equal (S105). When the impedance values Z1 and Z2 are equal (S105-Yes), the processor 41 performs control to maintain, with time, the voltage values V1 and V2 at a set value Vb. Accordingly, the processor 41 sets the voltage values V1 and V2 to the same value (S106). In the process of S107 to be described later, if control to set the voltage values V1 and V2 to values different from each other is performed, the control is switched to control to maintain, with time, each of the voltage values V1 and V2 at the set value Vb. Then, the process proceeds to S108.

If the impedance values Z1 and Z2 are different from each other (S105-No), the processor 41 sets the smaller impedance value of the calculated impedance values Z1 and Z2 as Zs, and sets the greater impedance value as Zh. Therefore, the impedance value Zh is greater than the impedance value Zs, i.e., Zs<Zh. The impedance value Zs is one of the impedance values Z1 and Z2, and the impedance value Zh is the other of the impedance values Z1 and Z2.

A voltage value of the electric circuit (the one of 58 and 68) corresponding to the impedance value Zs is set as Vs, and a voltage value of the electric circuit (the other of 58 and 68) corresponding to the impedance value Zh is set as Vh. The voltage value Vs is one of the voltage values V1 and V2, and the voltage value Vh is the other of the voltage values V1 and V2. The processor 41 controls the outputs from the high-frequency power sources 51 and 61 to maintain, with time, the voltage value Vh at the set value

Vc and to maintain, with time, the voltage value Vs at the set value Vd. The set values Vc and Vd are stored in, for example, the storage medium 42. Here, the set value Vd is greater than the set value Vc. Therefore, Vd>Vc. The set values Vc and Vd are, for example, greater than the set value Va for impedance detection. When the voltage value Vs becomes the set value Vd greater than the set value Vc, the voltage value Vs becomes greater than the voltage value Vh (S107). Then, the process proceeds to S108.

As described above, when the thickness T1 of the first portion B1 is larger (thicker) than the thickness T2 of the second portion B2, the impedance value Z1 is smaller than the impedance value Z2. Thus, Zs=Z1 and Zh=Z2. Further, Vs=V1 and Vh=V2. Therefore, the impedance value Zs is the impedance value (Z1) of the electric circuit (58) that passes the high-frequency current through the thick portion (B1) of the blood vessel B. The impedance value Zh is the impedance value (Z2) of the electric circuit (68) that passes the high-frequency current through the thin portion (B2) of the blood vessel B. The voltage value Vs is the voltage value (V1) of the output voltage to the electric circuit (58) that passes the high-frequency current through the thick portion (B1) of the blood vessel B. The voltage value Vh is the voltage value (V2) of the output voltage to the electric circuit (68) that passes the high-frequency current through the thin portion (B2) of the blood vessel B.

In the process of S108, the processor 41 determines whether or not the reference value Za is equal to or greater than a threshold Zth1. The threshold Zth1 is stored in, for example, the storage medium 42.

If the reference value Za is smaller than the threshold Zth1 (S108-No), the process returns to S103, and the processor 41 sequentially executes the processes of S103 and the subsequent steps. Thus, the processes of S103 and the subsequent steps are repeatedly executed until it is determined in the process of S108 that the reference value Za is equal to or greater than the threshold Zth1.

If the reference value Za is equal to or greater than the threshold Zth1 (S108-Yes), the processor 41 finishes the output control in the first output mode and switches the output control to output control in the second output mode. In the second output mode, the processor 41 controls the outputs from the high-frequency power sources 51 and 61 to maintain, with time, the voltage values V1 and V2 at a set value Ve (S109). The set value Ve is an output value for carefully and slowly coagulating the grasped tissue. The set value Ve is, for example, a value greater than the set value Va and smaller than the set values Vb, Vc, and Vd. The set value Ve is stored in, for example, the storage medium 42.

In the second output mode, the processor 41 then determines whether or not the reference value Za is equal to or greater than a threshold Zth2 (S110). The threshold Zth2 is, for example, an average value of the impedance values Z1 and Z2 in a state in which the tissue is sufficiently coagulated. The threshold Zth2 is stored in, for example, the storage medium 42. The process of S110 is repeatedly executed until it is determined that the reference value Za is equal to or greater than the threshold Zth2.

If the reference value Za is equal to or greater than the threshold Zth2 (S110-Yes), the processor 41 finishes the output control in the second output mode, and stops the output of the electric energy from the high-frequency power sources 51 and 61 of the controller 3 to the treatment instrument 2. When the output of the electric energy from the controller 3 to the treatment instrument 2 is stopped, the application of the high-frequency current to the blood vessel B is finished, and the coagulation treatment of the blood vessel B is finished.

In the present embodiment, the average value of the impedance values Z1 and Z2 is used as the reference value Za, but the reference value Za is not limited thereto. For example, one of the impedance values Z1 and Z2 may be used as the reference value Za.

As described above, when the thickness T1 of the first portion B1 and the thickness T2 of the second portion B2 are different in the blood vessel B, the impedance values Z1 and Z2 are different from each other. In the present embodiment, in S105, the processor 41 determines whether or not the impedance values Z1 and Z2 are different from each other. In a case where the impedance values Z1 and Z2 are different from each other, it is determined that the thickness T1 of the first portion B1 and the thickness T2 of the second portion B2 in the blood vessel B are different from each other. Then, based on the determination that the thickness T1 of the first portion B1 and the thickness T2 of the second portion B2 are different from each other, the process of S107 is performed.

In the present embodiment, in the process of S107, the processor 41 sets the voltage value Vs to be greater than the voltage value Vh. Thus, the voltage value (one of V1 and V2) of the output voltage to the electric circuit (one of 58 and 68) that passes the high-frequency current through the thick portion (one of B1 and B2) of the blood vessel B becomes greater than the voltage value (the other of V1 and V2) of the output voltage to the electric circuit (the other of 58 and 68) that passes the high-frequency current through the thin portion (the other of B1 and B2) of the blood vessel B. Thereby, the high-frequency current flowing through the thick portion (one of B1 and B2) of the blood vessel B becomes larger than the high-frequency current flowing through the thin portion (the other of B1 and B2) of the blood vessel B. Therefore, an energy amount of the electric energy applied to the thick portion (one of B1 and B2) of the blood vessel B becomes larger than an energy amount of the electric energy applied to the thin portion (the other of B1 and B2) of the blood vessel B.

As described above, in the present embodiment, the energy amount of the electric energy applied to the thick portion is larger than the energy amount of the electric energy applied to the thin portion. Accordingly, a coagulation rate (contraction rate) at the thick portion is higher than a coagulation rate (contraction rate) at the thin portion. Then, when the coagulation rate (contraction rate) of the thick portion increases, a difference in thickness between the thick portion and the thin portion becomes small. When the difference in thickness between the thick portion and the thin portion becomes small, a difference between the impedance value of the electric circuit that passes the high-frequency current through the thick portion and the impedance value of the electric circuit that passes the high-frequency current through the thin portion becomes small.

In the present embodiment, the processor 41 equalizes the voltage values V1 and V2 in a state in which the difference between the impedance values Z1 and Z2 becomes small. Thus, the energy amount of the electric energy to be supplied to the living tissue becomes equal. In a state in which the difference in thickness of the grasped living tissue becomes small, the energy amount of the electric energy to be supplied becomes equal, whereby the living tissue can be coagulated uniformly.

### Second Embodiment

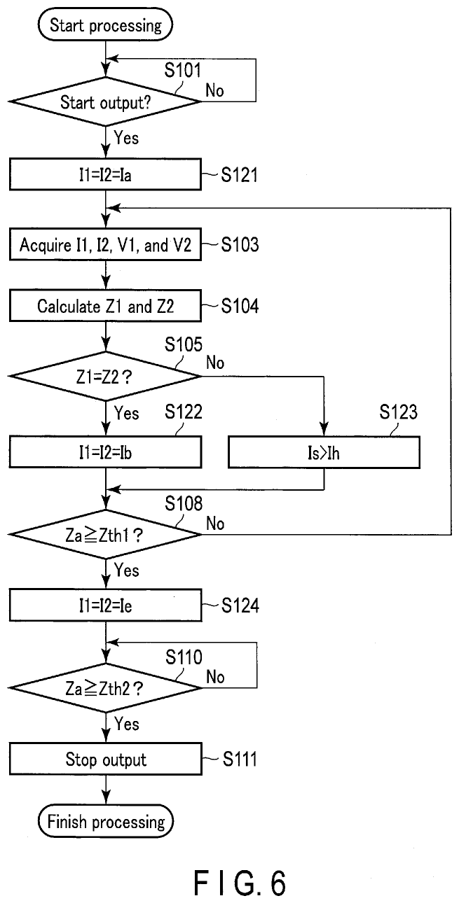

A second embodiment will be described with reference to FIG. 6. In the present embodiment, the configuration of the first embodiment is modified as follows. The same components as those of the first embodiment are denoted by the same reference signs, and a description thereof will be omitted.

FIG. 6 is a flowchart showing processing performed by the processor 41 when a coagulation treatment of a treatment target is performed using the treatment system 1 of the present embodiment. As shown in FIG. 6, in the present embodiment, the processor 41 performs control to maintain, with time, each of the current values I1 and I2 at a predetermined value (set value) by controlling the output from the high-frequency power sources 51 and 61 to the treatment instrument 2.

If an electric signal indicating that an output operation is input at the operation button 19 is detected (S101-Yes), the processor 41 performs control to maintain the current values I1 and I2 at a set value Ia (S121). Thus, the current values I1 and I2 become the same value. The set value Ia is an output value for detecting the thickness of the grasped tissue. The set value Ia is stored in, for example, the storage medium 42.

In the present embodiment, if the impedance values Z1 and Z2 are equal to each other in the determination of S105 (S105-Yes), the processor 41 performs control to maintain, with time, the current values I1 and I2 at a set value Ib. Accordingly, the processor 41 sets the current values I1 and I2 to the same value (S122). In the process of S123 to be described later, if control to maintain the current values I1 and I2 at values different from each other is performed, the control is switched to control to maintain each of the current values I1 and I2 at the set value Ib. Then, the process proceeds to S108.

If the impedance values Z1 and Z2 are different from each other (S105-No), a current value of the electric circuit (the one of 58 and 68) corresponding to the impedance value Zs is set as Is, and a current value of the electric circuit (the other of 58 and 68) corresponding to the impedance value Zh is set as Ih. The current value Is is one of the current values I1 and I2, and the current value Ih is the other of the current values I1 and I2.

As described above, for example, when the thickness T1 of the first portion B1 is larger (thicker) than the thickness T2 of the second portion B2, the impedance value Z1 is smaller than the impedance value Z2. Thus, Zs=Z1 and Zh=Z2. Then, Is=I1 and Ih=12. Therefore, the current value Is is the current value (I1) of the output current to the thick portion (B1) of the blood vessel B. The current value Ih is the current value (I2) of the output current to the thin portion (B2) of the blood vessel B.

In the present embodiment, if the impedance values Z1 and Z2 are different from each other in S105 (S105-No), the processor 41 controls the outputs from the high-frequency power sources 51 and 61 to maintain, with time, the current value Ih at a set value Ic and to maintain, with time, the current value Is at a set value Id. The set values Ic and Id are stored in, for example, the storage medium 42. Here, the set value Id is a value greater than the set value Ic. Therefore, Id>Ic. In addition, the set values Ic and Id are, for example, greater than the set value Ia for impedance detection. When the current value Is becomes the set value Id greater than the set value Ic, the current value Is becomes greater than the current value Ih (S123). Then, the process proceeds to S108. When the reference value Za is equal to or greater than the threshold Zth1 (S108-Yes), the processor 41 finishes the output control in the first output mode and switches the output control to the output control in the second output mode. In the second output mode, the processor 41 controls the outputs from the high-frequency power sources 51 and 61 to maintain, with time, the current values I1 and I2 at a set value Ie (S124). The set value Ie is an output value for carefully and slowly coagulating the grasped tissue. The set value Ie is, for example, a value greater than the set value Ia and smaller than the set values Ib, Ic, and Id.

In the present embodiment, in the process of S123, the processor 41 sets the current value Is to be greater than the current value Ih. Thus, the current value (one of I1 and I2) of the output current supplied to the thick portion (one of B1 and B2) of the blood vessel B is greater than the current value (the other of I1 and I2) of the output current supplied to the thin portion (the other of B1 and B2) of the blood vessel B. Therefore, the energy amount of the electric energy applied to the thick portion (one of B1 and B2) of the blood vessel B becomes larger than the energy amount of the electric energy applied to the thin portion (the other of B1 and B2) of the blood vessel B.

As described above, in the present embodiment, the energy amount of the electric energy supplied to the grasped living tissue is adjusted by controlling the current values I1 and I2. Then, the energy amount of the electric energy applied to the thick tissue is made larger than the amount of electric energy applied to the thin tissue. Therefore, similarly to the first embodiment, the living tissue can be uniformly coagulated.

### Third Embodiment

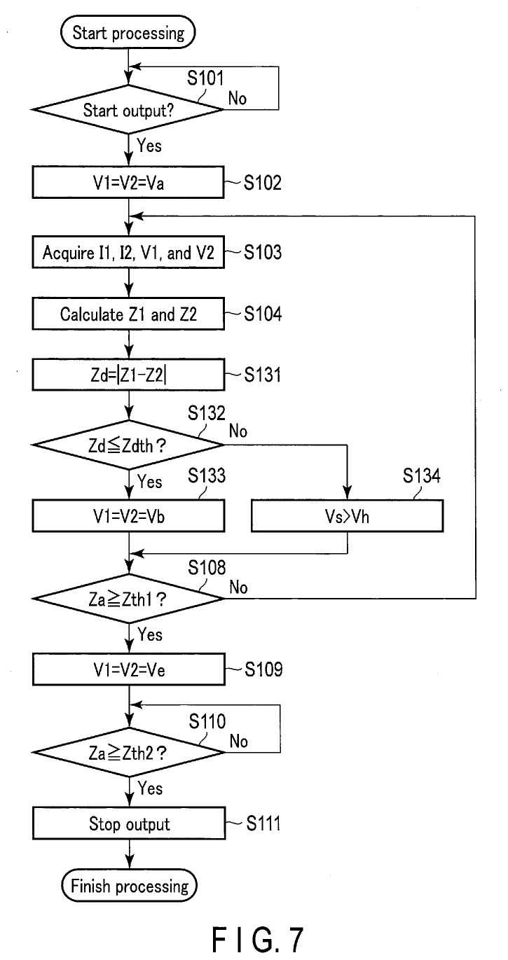

A third embodiment will be described with reference to FIG. 7. In the present embodiment, the configuration of the first embodiment is modified as follows. The same components as those of the first embodiment are denoted by the same reference signs, and a description thereof will be omitted.

FIG. 7 is a flowchart showing processing performed by the processor 41 when a coagulation treatment of a treatment target is performed using the treatment system 1 of the present embodiment. As shown in FIG. 7, in the present embodiment, after the impedance values Z1 and Z2 of the electric circuits 58 and 68 are calculated in S104, the processor 41 calculates a difference value Zd between the impedance values of the electric circuits 58 and 68 (S131). The difference value Zd is an absolute value of a difference between the impedance value Z1 and the impedance value Z2. The difference value Zd is calculated by the equation “Zd=|Z1−Z2|”.

Next, the processor 41 determines whether or not the impedance difference value Zd is equal to or less than a threshold Zdth (S132). The threshold Zdth is stored in, for example, the storage medium 42. If the difference value Zd is equal to or less than the threshold Zdth (S132-Yes), the processor 41 performs control to maintain, with time, the voltage values V1 and V2 at the set value Vb by controlling the outputs from the high-frequency power sources 51 and 61. Accordingly, the processor 41 sets the voltage values V1 and V2 to the same value (S133). In the process of S134 to be described later, if control in which the voltage values V1 and V2 are maintained at values different from each other is performed, the control is switched to control in which each of the voltage values V1 and V2 is maintained at the set value Vb. Then, the process proceeds to S108.

If the difference value Zd is greater than the threshold Zdth (S132-No), the processor 41 sets the impedance values Zs and Zh and the voltage values Vs and Vh in the same manner as in the first embodiment. Then, the processor 41 controls the outputs from the high-frequency power sources 51 and 61 to maintain, with time, the voltage value Vh at the set value Vc and to maintain, with time, the voltage value Vs at the set value Vd. The set values Vc and Vd are stored in, for example, the storage medium 42. Here, the set value Vd is greater than the set value Vc. Therefore, Vd>Vc. The set values Vc and Vd are, for example, greater than the set value Va for impedance detection. When the voltage value Vs becomes the set value Vd greater than the set value Vc, the voltage value Vs becomes greater than the voltage value Vh (S134). Then, the process proceeds to S108.

As described above, when the thickness T1 of the first portion B1 and the thickness T2 of the second portion B2 are different in the blood vessel B, the impedance values Z1 and Z2 are different from each other. In the present embodiment, in S131, the processor 41 determines whether or not the difference value Zd between the impedance values Z1 and Z2 is smaller than the threshold value Zdth. If the difference value Zd is smaller than the threshold Zdth, it is determined that the difference between the thickness T1 of the first portion B1 and the thickness T2 of the second portion B2 is smaller than a predetermined value. In this case, it is determined that the thickness of the grasped living tissue is uniform.

If the difference value Zd is equal to or greater than the threshold Zdth, it is determined that the difference between the thickness T1 of the first portion B1 and the thickness T2 of the second portion B2 is equal to or greater than the predetermined value. In this case, it is determined that the thickness of the grasped living tissue is not uniform. Then, the processor 41 makes the energy amount of the electric energy applied to the thick tissue larger than the energy amount of the electric energy applied to the thin tissue in the same manner as in the above-described embodiments, etc. by setting the voltage value Vs to be greater than the voltage value Vh. Thus, the living tissue can be uniformly coagulated in the same manner as in the first embodiment, etc.

### Fourth Embodiment

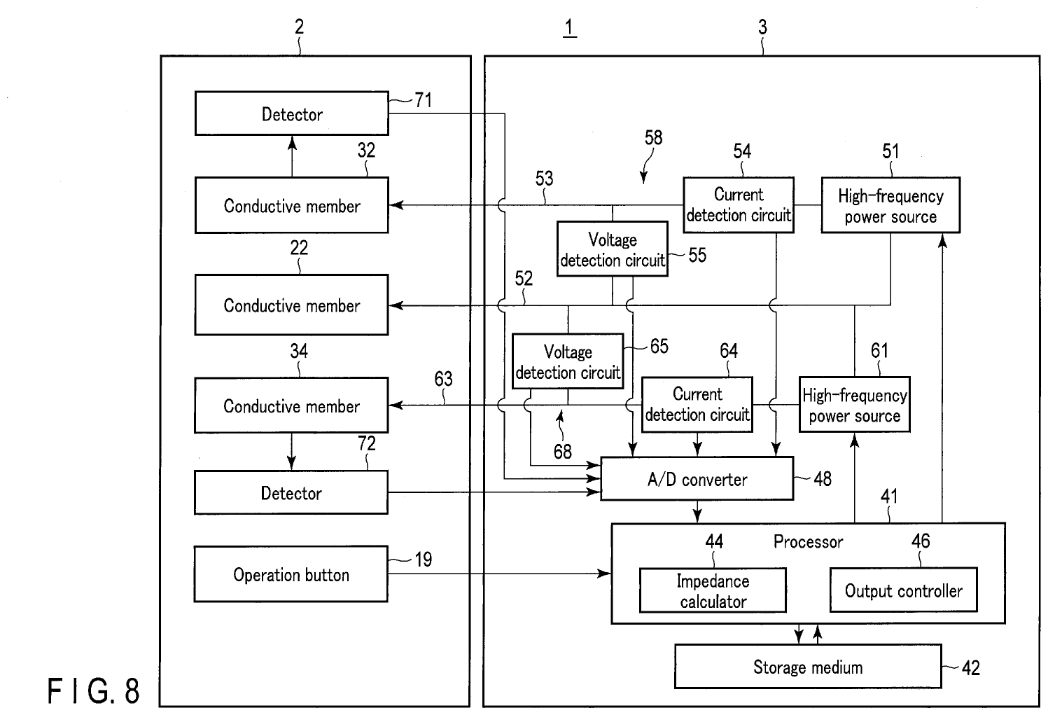

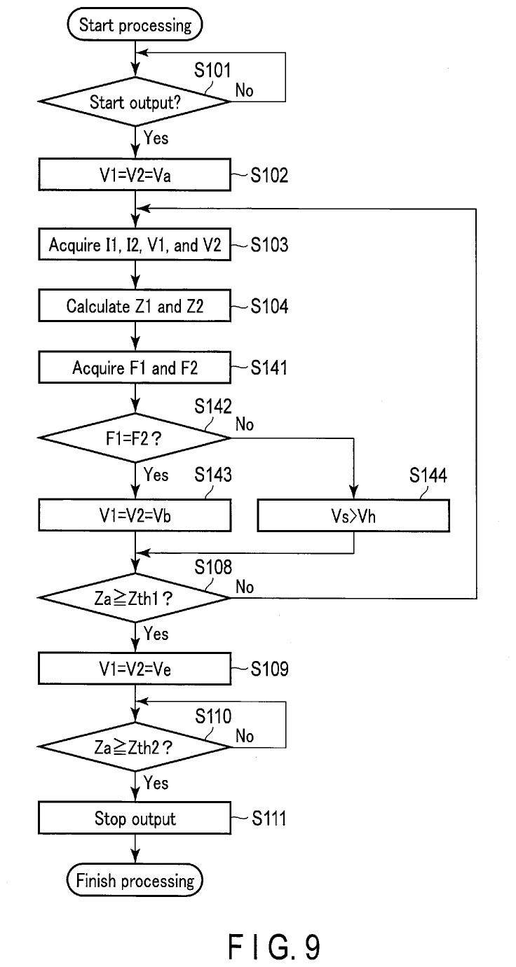

A fourth embodiment will be described with reference to FIGS. 8 and 9. In the present embodiment, the configuration of the first embodiment is modified as follows. The same components as those of the first embodiment are denoted by the same reference signs, and a description thereof will be omitted.

FIG. 8 is a block diagram showing a control configuration in the treatment system 1 of the present embodiment. As shown in FIG. 8, the treatment system 1 of the present embodiment includes detectors 71 and 72. The detector (first detector) 71 is attached to the conductive member 32 of the second grasping piece 14. The detector 71 detects a parameter (first parameter) relating to the thickness of the treatment target grasped between the conductive members 22 and 32 in the first region D1. That is, the detector 71 detects a parameter relating to the thickness of a portion of the treatment target that is disposed in the first region D1.

The detector (second detector) 72 is attached to the conductive member 34 of the second grasping piece 14. The detector 72 detects a parameter (second parameter) relating to the thickness of the treatment target grasped between the conductive members 22 and 34 in the second region D2. That is, the detector 72 detects a parameter relating to the thickness of a portion of the treatment target that is disposed in the second region D2.

An analog signal detected by each of the detectors 71 and 72 is converted into a digital signal by the A/D converter 48. The converted digital signal is transmitted to the processor 41.

The processor 41 acquires the parameters relating to the thickness of the grasped treatment target from the detectors 71 and 72. The processor 41 acquires, from the detector 71, the parameter (first parameter) relating to the thickness of the treatment target at the portion grasped in the first region D1, and acquires, from the detector 72, the parameter (second parameter) relating to the thickness of the treatment target at the portion grasped in the second region D2. Then, the output controller 46 of the processor 41 controls the output of the electric energy supplied from the high-frequency power sources 51 and 61 to the treatment instrument 2 based on the acquired parameters relating to the thickness of the treatment target.

In the present embodiment, pressure sensors are used as the detectors 71 and 72. The detector (pressure sensor) 71 detects a pressure value F1 acting on the conductive member 32 from the living tissue grasped in the first region D1. The detector (pressure sensor) 72 detects a pressure value F2 acting on the conductive member 34 from the living tissue grasped in the second region D2. Then, the processor 41 acquires the pressure values F1 and F2 detected by the detectors 71 and 72 as the parameters relating to the thickness of the grasped living tissue. The processor 41 controls the output of the electric energy to be supplied to the conductive members 22 32, and 34 based on the acquired pressure values F1 and F2.

When the blood vessel B is grasped as the treatment target between the grasping pieces 13 and 14, the thickness of the first portion B1 of the blood vessel B and the thickness of the second portion B2 of the blood vessel B may be different from each other. In this case, the pressure value F1 acting on the conductive member 32 from the first portion B1 and the pressure value F2 acting on the conductive member 34 from the second portion B2 are different from each other. For example, when the thickness T1 of the first portion B1 is smaller than the thickness T2 of the second portion B2, the pressure value F1 acting on the conductive member 32 is smaller than the pressure value F2 acting on the conductive member 34. In addition, for example, when the thickness T1 of the first portion B1 grasped in the first region D1 is larger than the thickness T2 of the second portion B2 grasped in the second region D2, the pressure value F1 acting on the conductive member 32 is greater than the pressure value F2 acting on the conductive member 34.

FIG. 9 is a flowchart showing processing performed by the processor 41 when a coagulation treatment of a treatment target is performed using the treatment system 1 of the present embodiment. As shown in FIG. 9, in the present embodiment, after the output of the electric energy is started, the processor 41 acquires the pressure values F1 and F2 detected by the detectors (pressure sensors) 71 and 72 as parameters relating to the thickness of the grasped living tissue (S141).

Next, the processor 41 determines whether or not the pressure values F1 and F2 are equal to each other (S142). If the pressure values F1 and F2 are equal (S142-Yes), the processor 41 performs control to maintain, with time, the voltage values V1 and V2 at the set value Vb. Accordingly, the processor 41 sets the voltage values V1 and V2 to the same value (S143). In the process of S144 to be described later, if control in which the voltage values V1 and V2 are maintained at values different from each other is performed, the control is switched to control to maintain each of the voltage values V1 and V2 at the set value Vb. Then, the process proceeds to S108.

If the pressure values F1 and F2 are different from each other (S142-No), the processor 41 sets the greater pressure value of the acquired pressure values F1 and F2 as Fs and sets the smaller pressure value as Fh. The pressure value Fh is smaller than the pressure value Fs. Therefore, Fs>Fh. The pressure value Fs is one of the pressure values F1 and F2, and the pressure value Fh is the other of the pressure values F1 and F2.

Further, a voltage value of an output voltage to the counter electrode (the one of 32 and 34) corresponding to the pressure value Fs is set as Vs, and a voltage value of an output voltage to the counter electrode (the other of 32 and 34) corresponding to the pressure value Fh is set as Vh. The voltage value Vs is one of the voltage values V1 and V2, and the voltage value Vh is the other of the voltage values V1 and V2. The processor 41 controls the outputs from the high-frequency power sources 51 and 61 to maintain, with time, the voltage value Vh at the set value Vc and to maintain, with time, the voltage value Vs at the set value Vd. The set values Vc and Vd are stored in, for example, the storage medium 42. Here, the set value Vd is a value greater than the set value Vc. Therefore, Vd >Vc. The set values Vc and Vd are, for example, greater than the set value Va for impedance detection. When the voltage value Vs becomes the set value Vd greater than the set value Vc, the voltage value Vs becomes greater than the voltage value Vh (S144). Then, the process proceeds to S108.

As described above, for example, when the thickness T1 of the first portion B1 is larger (thicker) than the thickness T2 of the second portion B2, the pressure value F1 is greater than the pressure value F2. Thus, Fs=F1 and Fh=F2. Then, Vs=V1 and Vh=V2. Therefore, the voltage value Vs is the voltage value (V1) of the output voltage to the thick portion (B1) of the blood vessel B, and the voltage value Vh is the voltage value (V2) of the output voltage to the thin portion (B2) of the blood vessel B.

As described above, when the thickness T1 of the first portion B1 and the thickness T2 of the second portion B2 are different in the blood vessel B, the pressure values F1 and F2 are different from each other. In the present embodiment, in S142, the processor 41 determines whether or not the pressure values F1 and F2 are different from each other. If the pressure values F1 and F2 are different from each other, it is determined that the thickness T1 of the first portion B1 and the thickness T2 of the second portion B2 are different from each other in the blood vessel B. Then, based on the determination that the thickness T1 of the first portion B1 and the thickness T2 of the second portion B2 are different from each other, the process of S144 is performed.

Also in the present embodiment, in the process of S144, the processor 41 sets the voltage value Vs to be greater than the voltage value Vh. Thus, also in the present embodiment, the energy amount of the electric energy applied to the thick portion (one of B1 and B2) of the blood vessel B is larger than the energy amount of the electric energy applied to the thin portion (the other of B1 and B2) of the blood vessel B.

### First Modification of Fourth Embodiment

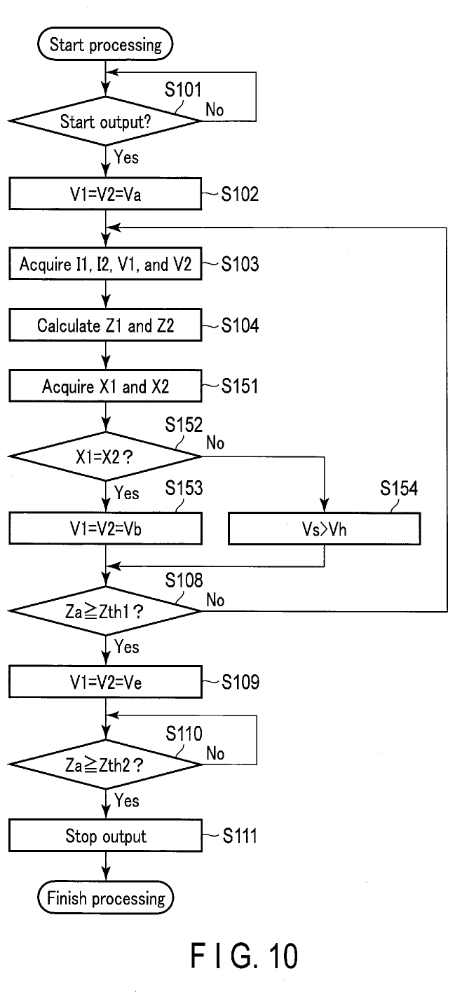

A first modification of the present embodiment will be described with reference to FIG. 10. In this modification, the configuration of the fourth embodiment is modified as follows. The same components as those of the fourth embodiment are denoted by the same reference signs, and a description thereof will be omitted.

In the present modification, displacement meters are used as the detectors 71 and 72. The displacement meter is, for example, a strain gauge. The detector (displacement meter) 71 detects displacement (strain) X1 generated in the conductive member 32 when the treatment target is grasped.

The detector (displacement meter) 72 detects displacement (strain) X2 generated in the conductive member 34 when the treatment target is grasped. The processor 41 acquires the displacements X1 and X2 generated in the conductive members 32 and 34 as parameters relating to the thickness of the grasped treatment target. Then, the processor 41 adjusts the outputs of the electric energy to be supplied to the conductive members 22, 32, and 34 based on the acquired displacements X1 and X2.

When the blood vessel B is grasped as the treatment target between the grasping pieces 13 and 14, the thickness of the first portion B1 of the blood vessel B and the thickness of the second portion B2 of the blood vessel B may be different from each other. In this case, the displacement X1 generated in the conductive member 32 and the displacement X2 generated in the conductive member 34 are different from each other. For example, when the thickness T1 of the first portion B1 is larger than the thickness T2 of the second portion B2, the displacement X1 generated in the conductive member 32 is larger than the displacement X2 generated in the conductive member 34. In addition, for example, when the thickness T1 of the first portion B1 grasped in the first region D1 is smaller than the thickness T2 of the second portion B2 grasped in the second region D2, the displacement X1 generated in the conductive member 32 is smaller than the displacement X2 generated in the conductive member 34.

FIG. 10 is a flowchart showing processing performed by the processor 41 when a coagulation treatment of a treatment target is performed using the treatment system 1 of the present modification. As shown in FIG. 10, in the present modification, after the output of the electric energy is started, the processor 41 acquires the displacements X1 and X2 detected by the detectors (displacement meters) 71 and 72 as parameters relating to the thickness of the grasped tissue (S151).

Next, the processor 41 determines whether or not the displacements X1 and X2 are equal to each other (S152). If the displacements X1 and X2 are equal (S152-Yes), the processor 41 performs control to maintain, with time, the voltage values V1 and V2 at the set value Vb. Accordingly, the processor 41 sets the voltage values V1 and V2 to the same value (S153). In the process of S154 to be described later, if control to maintain the voltage values V1 and V2 at values different from each other is performed, the control is switched to control to maintain each of the voltage values V1 and V2 at the set value Vb. Then, the process proceeds to S108.

If the displacements X1 and X2 are different from each other (S152-No), the processor 41 sets the larger one of the displacements X1 and X2 as Xs and the smaller one as Xh. The displacement Xh is smaller than the displacement Xs. Therefore, Xs >Xh. The displacement Xs is one of the displacements X1 and X2, and the displacement Xh is the other of the displacements X1 and X2.

Further, a voltage value of an output voltage to the counter electrode (the one of 32 and 34) corresponding to the displacement Xs is set as Vs, and a voltage value of an output voltage to the counter electrode (the other of 32 and 34) corresponding to the displacement Xh is set as Vh. The voltage value Vs is one of the voltage values V1 and V2, and the voltage value Vh is the other of the voltage values V1 and V2. The processor 41 controls the outputs from the high-frequency power sources 51 and 61 to maintain, with time, the voltage value Vh at the set value Vc and maintain, with time, the voltage value Vs at the set value Vd. The set values Vc and Vd are stored in, for example, the storage medium 42. Here, the set value Vd is a value greater than the set value Vc. Therefore, Vd>Vc. In addition, the set values Vc and Vd are, for example, greater than the set value Va for impedance detection. When the voltage value Vs becomes the set value Vd greater than the set value Vc, the voltage value Vs becomes greater than the voltage value Vh (S154). Then, the process proceeds to S108.

As described above, for example, when the thickness T1 of the first portion B1 is larger (thicker) than the thickness T2 of the second portion B2, the displacement X1 is larger than the displacement X2. Thus, Xs=X1 and Xh=X2. Then, Vs=V1 and Vh=V2. Therefore, the voltage value Vs is the voltage value (V1) of the output voltage to the electric circuit (58) that forms the thick portion (B1) of the blood vessel B. The voltage value Vh is the voltage value (V2) of the output voltage to the electric circuit (68) that forms the thin portion (B2) of the blood vessel B. As described above, when the thickness T1 of the first portion B1 and the thickness T2 of the second portion B2 are different in the blood vessel B, the displacements X1 and X2 are different from each other. In the present embodiment, in S152, the processor 41 determines whether or not the displacements X1 and X2 are different from each other. If the displacements X1 and X2 are different from each other, it is determined that the thickness T1 of the first portion B1 and the thickness T2 of the second portion B2 are different from each other in the blood vessel B. Then, based on the determination that the thickness T1 of the first portion B1 and the thickness T2 of the second portion B2 are different from each other, the process of S154 is performed.

Also in the present embodiment, in the process of S154, the processor 41 sets the voltage value Vs to be greater than the voltage value Vh. Thus, also in the present embodiment, the energy amount of the electric energy applied to the thick portion (one of B1 and B2) of the blood vessel B is larger than the energy amount of the electric energy applied to the thin portion (the other of B1 and B2) of the blood vessel B.

### Fifth Embodiment

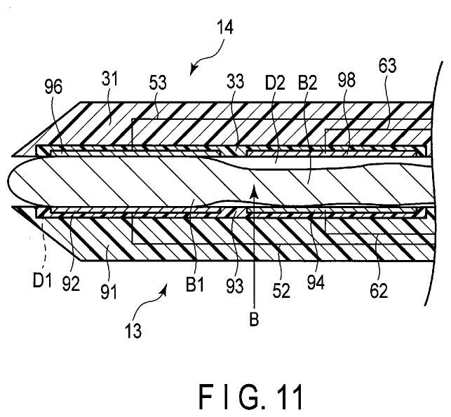

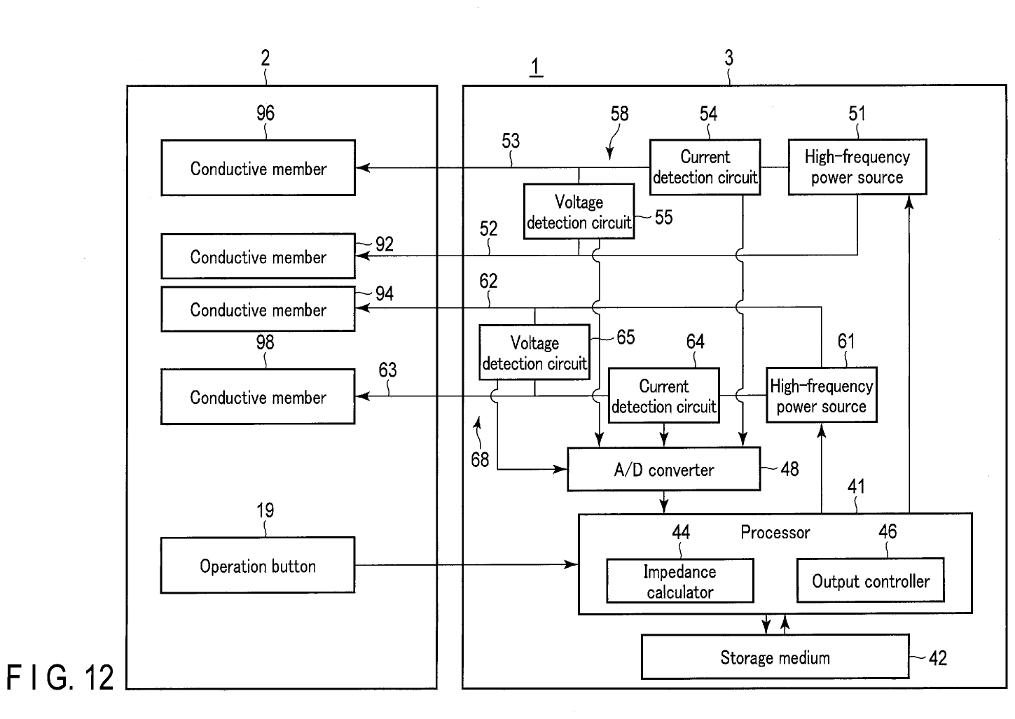

A fifth embodiment will be described with reference to FIGS. 11 and 12. In the present embodiment, the configuration of the first embodiment is modified as follows. The same components as those of the first embodiment are denoted by the same reference numerals, and a description thereof will be omitted.

FIG. 11 is a diagram showing a configuration of the grasping pieces 13 and 14 in the present embodiment. As shown in FIG. 11, in the present embodiment, the grasping piece 13 includes a support 91. The support 91 extends along the longitudinal axis C in a state in which the grasping piece 13 is closed relative to the grasping piece 14. A proximal end of the support 91 is attached to the distal end of the shaft 5. The support 91 is formed of, for example, a resin material having electrical insulation properties.

Conductive members 92 and 94 are attached to the support 91 from the grasping piece 14 side. Each of the conductive members 92 and 94 faces the treatment surface 18 of the second grasping piece 14, and forms a part of the treatment surface 17 of the grasping piece 13. Each of the conductive members 92 and 94 has conductivity. The conductive members 92 and 94 are made of, for example, a metal such as stainless steel. The conductive members 92 and 94 are separated from each other in the longitudinal direction. The conductive member 92 and the conductive member 94 are electrically insulated from each other in the longitudinal direction by the support 91 and an abutment member 93.

In the grasping piece 14, conductive members 96 and 98 are attached to a support 31 from the grasping piece 13 side. The conductive member 96 faces the conductive member 92 of the grasping piece 13. The conductive member 98 faces the conductive member 94 of the grasping piece 13. Therefore, each of the conductive members 96 and 98 faces the treatment surface 17 of the first grasping piece 13, and forms a part of the treatment surface 18 of the grasping piece 14. Each of the conductive members 96 and 98 has conductivity. Each of the conductive members 96 and 98 is made of, for example, a metal such as stainless steel. The conductive members 96 and 98 are separated from each other in the longitudinal direction. The conductive member 92 and the conductive member 94 are electrically insulated from each other in the longitudinal direction by the support 31 and an abutment member 33.

In the present embodiment, between the treatment surfaces 17 and 18, a space formed between the conductive members 92 and 96 is referred to as a first region D1, and a space formed between the conductive members 94 and 98 is referred to as a second region D2. In the present embodiment, the positions of the first region D1 and the second region D2 in the longitudinal direction are different from each other.

In the treatment system 1, the high-frequency power source 51 is electrically connected to the conductive member 92 via the electric path 52 formed of electric wiring, etc. Further, the high-frequency power source (first power source) 51 is electrically connected to the conductive member 96 via the electric path 53 formed of electric wiring, etc. The high-frequency power source (second power source) 61 is electrically connected to the conductive member 94 via the electric path 62 formed of electric wiring, etc. Then, the high-frequency power source (second power source) 61 is electrically connected to the conductive member 98 via the electric path 63 formed of electric wiring, etc. Each of the electric paths 52, 53, 62, and 63 extends through the inside of the cable 7, the inside of the housing 4, and the inside of the shaft 5. When the high-frequency power is output from the high-frequency power source 51, a voltage (potential difference) is applied between the conductive members 92 and 96. In addition, when the high-frequency power is output from the high-frequency power source 61, a voltage (potential difference) is applied between the conductive members 94 and 98. Therefore, when the high-frequency power is supplied from the high-frequency power sources 51 and 61, the conductive member (first reference electrode) 92, conductive member (second reference electrode) 94, conductive member (first counter electrode) 96, and conductive member (second counter electrode) 98 function as electrodes different from each other.

In the present embodiment, the electric circuit 58 is formed by the high-frequency power source 51, electric path 52, conductive member 92, conductive member 96, and electric path 53, and the electric circuit 68 is formed by the high-frequency power source 61, electric path 62, conductive member 94, conductive member 98, and electric path 63.

FIG. 11 is a diagram showing a state in which the blood vessel B is grasped as a treatment target between the pair of grasping pieces 13 and 14. Here, in the blood vessel B, a portion grasped between the conductive members 92 and 96 is referred to as the first portion B1, and a portion grasped between the conductive members 94 and 98 is referred to as the second portion B2. The first portion B1 is a portion disposed in the first region D1, and the second portion B2 is a portion disposed in the second region D2. That is, the first portion B1 is a portion disposed between the first reference electrode (92) and the first counter electrode (96), and the second portion B2 is a portion disposed between the second reference electrode (94) and the second counter electrode (98). In the present embodiment, the positions of the first portion B1 and the second portion B2 in the longitudinal direction are different from each other.

For example, as shown in FIG. 11, the thickness T1 of the first portion B1 may be larger (thicker) than the thickness T2 of the second portion B2. In this case, the thickness of the grasped blood vessel B is different in the longitudinal direction. Then, the impedance value Z1 of the electric circuit 58 that passes the high-frequency current through the first portion B1 of the blood vessel B is smaller than the impedance value Z2 of the electric circuit that passes the high-frequency current through the second portion B2 of the blood vessel B.

Further, for example, in a case where the thickness T1 of the first portion B1 is smaller (thinner) than the thickness T2 of the second portion B2 in the blood vessel B, the impedance value Z1 of the electric circuit 58 that passes the high-frequency current through the first portion B1 is greater than the impedance value Z2 of the electric circuit 68 that passes the high-frequency current through the second portion B2.

Also in the present embodiment, the processor 41 performs the same processing as that in the first embodiment, and sets the voltage value Vs to be greater than the voltage value Vh in the process of S107. Accordingly, the energy amount of the electric energy applied to the thick portion (one of B1 and B2) of the blood vessel B becomes larger than the energy amount of the electric energy applied to the thin portion (the other of B1 and B2) of the blood vessel B. In a state in which the difference between the impedance values Z1 and Z2 becomes small, the processor 41 equalizes the voltage values V1 and V2. Thus, the energy amount of the electric energy to be supplied to the living tissue becomes equal. In a state in which the difference in thickness of the grasped living tissue becomes small, the energy amount of the electric energy to be supplied becomes equal, whereby the living tissue can be coagulated uniformly.

### Sixth Embodiment

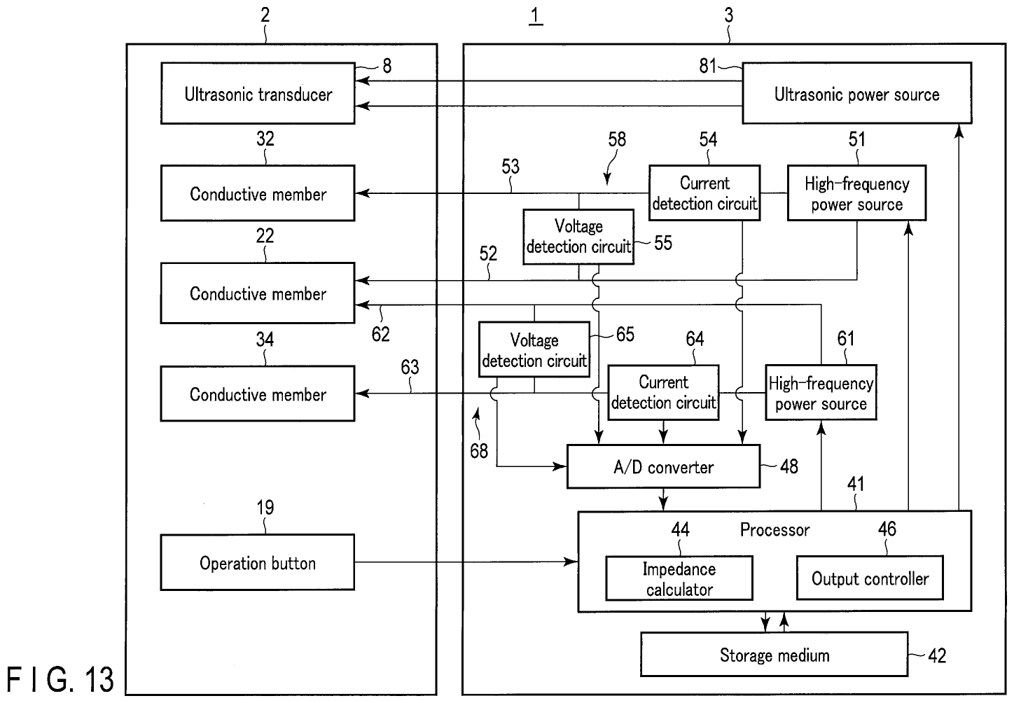

A sixth embodiment will be described with reference to FIG. 13. In the present embodiment, the configuration of the first embodiment is modified as follows. The same components as those of the first embodiment are denoted by the same reference signs, and a description thereof will be omitted.

FIG. 13 is a block diagram showing a control configuration in the treatment system 1 of the present embodiment. As shown in FIG. 13, in the present embodiment, the controller 3 includes an ultrasonic power source 81 in addition to the high-frequency power sources 51 and 61. The ultrasonic power source 81 includes a waveform generator, a conversion circuit, a transformer, etc., and converts electric power from a battery power source, an outlet power source, etc. into alternating-current electric power. An ultrasonic transducer 8 is provided inside the housing main body 10. The ultrasonic power source 81 is electrically connected to the ultrasonic transducer 8 via an electric path provided through the inside of the cable 7 and the inside of the housing 4. When electric energy (alternating-current electric power) is supplied from the ultrasonic power source 81 to the ultrasonic transducer 8, ultrasonic vibration is generated in the ultrasonic transducer 8.

A vibration transmitting member (ultrasonic probe) is detachably connected to a distal side of the ultrasonic transducer 8. The vibration transmitting member extends from the inside of the housing main body 10 to the distal side, passes through the inside of the shaft 5, and protrudes from the distal end of the shaft 5 to the distal side. The first grasping piece 13 is formed by a portion of the vibration transmitting member that protrudes from the shaft 5 to the distal side. The ultrasonic vibration generated by the ultrasonic transducer 8 is transmitted to the vibration transmitting member, and is transmitted to the distal end of the vibration transmitting member that forms the first grasping piece 13. Thus, the ultrasonic vibration is transmitted to the first grasping piece 13 as the treatment energy. When the ultrasonic vibration is transmitted to the first grasping piece 13 in a state in which the treatment target is grasped between the first grasping piece 13 and the second grasping piece 14, the ultrasonic vibration is applied to the treatment target as the treatment energy.

In the present embodiment, the output controller 46 of the processor 41 controls the supply of the electric energy for actuating the treatment instrument 2 to the treatment instrument 2 by controlling the output from the controller 3. The treatment instrument 2, by being supplied with the electric energy, applies at least one of the above-described high-frequency current and ultrasonic vibration as the treatment energy to the treatment target. For example, in a treatment of coagulating a treatment target such as a living tissue, the high-frequency current is applied to the treatment target as the treatment energy. Further, for example, in a treatment of coagulating and incising a treatment target such as a living tissue, both the high-frequency current and the ultrasonic vibration are applied to the treatment target as the treatment energy.

### Other Embodiments

In one embodiment, a variable resistance is provided in each of the electric circuits 58 and 68. The processor controls a resistance value of each of the variable resistances as a parameter relating to the output of the electric energy. The processor 41 controls an energy amount of electric energy applied to a grasped treatment target by controlling the resistance value of the variable resistance.

For example, the processor 41 sets the resistance value of the variable resistance provided in the electric circuit (one of 58 and 68) that passes the high-frequency current through the thick portion (one of B1 and B2) of the blood vessel B to be smaller than the resistance value of the variable resistance provided in the electric circuit (the other of 58 and 68) that passes the high-frequency current through the thin portion (the other of B1 and B2) of the blood vessel B. Thus, a current value of the high-frequency current flowing through the thick portion of the blood vessel B becomes greater than a current value of the high-frequency current flowing through the thin portion of the blood vessel B. Accordingly, the energy amount of the electric energy applied to the thick portion (one of B1 and B2) of the blood vessel B becomes larger than the energy amount of the electric energy applied to the thin portion (the other of B1 and B2) of the blood vessel B.

In one embodiment, when it is determined that the thickness T1 of the first portion B1 and the thickness T2 of the second portion B2 are different from each other, the processor 41 adjusts the voltage value Vh instead of the voltage value Vs to set the voltage value (Vh) of the electric circuit (the one of 58 and 68) that passes the high-frequency current through the thin portion (one of B1 and B2) of the blood vessel B to be smaller than the voltage value (Vs) of the electric circuit (the other of 58 and 68) that passes the high-frequency current through the thick portion (the other of B1 and B2) of the blood vessel B.

In one embodiment, three or more conductive members (counter electrodes) are provided in the grasping piece 14.

The conductive members are electrically insulated from one another. Each of the conductive members forms a part of the treatment surface 18, and positions thereof on the treatment surface 18 are different from one another. Three or more high-frequency power sources connected to the respective conductive members are provided in the controller 3. Three or more regions corresponding to the respective counter electrodes and different in position from one another are formed between the treatment surfaces 17 and 18. Also in this embodiment, when the thickness of a grasped tissue is different in each region, an amount of energy applied to a thick portion is made larger than an amount of energy applied to a thin portion, whereby the tissue can be coagulated uniformly.

In one embodiment, heat generated by a heater (heat source) is used as treatment energy. In this case, a heater (not shown) is provided in the end effector 6, and a heat power source (not shown) is provided in the controller 3. The heat power source supplies to the heater direct-current electric power or alternating-current electric power as electric energy for actuating the treatment instrument 2. As the electric energy is supplied to the heater, heat from the heater is applied to a treatment target.

In this embodiment, the treatment instrument 2, by being supplied with the electric energy, applies at least one of the above-described high-frequency current and heat to the treatment target as the treatment energy. For example, in a treatment of coagulating a treatment target such as a living tissue, the high-frequency current is applied to the treatment target as the treatment energy. Further, for example, in a treatment of coagulating and incising a treatment target such as a living tissue, both the high-frequency current and the heat are applied to the treatment target as the treatment energy.

In one embodiment, a cutter (cold cutter) is provided in the end effector 6 for incising a treatment target. In this case, a groove is formed on each of the treatment surface 17 of the first grasping piece 13 and the treatment surface 18 of the second grasping piece 14. On each of the treatment surfaces 17 and 18, the groove is provided at a center position in the width direction, and extends along the longitudinal direction (the extending direction of the grasping piece 14). Then, in a state in which the treatment target is grasped between the grasping pieces 13 and 14, the cutter is inserted into the aforementioned groove from the proximal side, whereby the grasped treatment target is incised.

Note that the present disclosure is not limited to the above embodiments, and can be modified in various ways without departing from the gist of the present disclosure in the implementation stage. Furthermore, each embodiment may be implemented by appropriate combinations thereof to a maximum extent; in which case a combined effect will be obtained. Moreover, the above embodiments include various stages, and various embodiments may be extracted by appropriate combinations of a plurality of disclosed configuration requirements.

1. A controller used together with an energy treatment instrument, the energy treatment including a first grasping piece, a second grasping piece configured to grasp a treatment target by opening and closing relative to the first grasping piece, a reference electrode provided in the first grasping piece, a first counter electrode provided in the second grasping piece, and a second counter electrode provided in the second grasping piece, the controller comprising:
a processor configured to:
acquire (i) a first parameter relating to a thickness of a first portion of the treatment target, the first portion grasped between the reference electrode and the first counter electrode, and (ii) a second parameter relating to a thickness of a second portion of the treatment target, the second portion grasped between the reference electrode and the second counter electrode;
calculate a difference value between the first parameter and the second parameter; and
control an amount of electric energy applied to one of the first portion or the second portion, which has a greater thickness, of the treatment target, such that the amount of electric energy applied is larger than an amount of electric energy applied to another one of the first portion or the second portion, which has a lesser thickness, of the treatment target when the calculated difference value is greater than a predetermined value.

2. The controller according to claim 1, further comprising:
a first power source configured to output electric energy to the first counter electrode; and
a second power source configured to output electric energy to the second counter electrode.

3. The controller according to claim 1, wherein the processor is configured to:
calculate, as the first parameter, a first impedance value in a first electric circuit configured to supply electric energy to the first counter electrode, and
calculate, as the second parameter, a second impedance value in a second electric circuit configured to supply electric energy to the second counter electrode.

4. The controller according to claim 3, wherein the processor is configured to:
determine that the thickness of the first portion of the treatment target is greater than the thickness of the second portion of the treatment target when the first impedance value is smaller than the second impedance value; and
determine that the thickness of the first portion of the treatment target is less than the thickness of the second portion of the treatment target when the first impedance value is greater than the second impedance value.

5. The controller according to claim 1, wherein:
the energy treatment instrument used together with the controller further includes:
a first detector provided on the first counter electrode and configured to detect the first parameter; and
a second detector provided on the second counter electrode and configured to detect the second parameter, and
the processor is configured to acquire the first parameter from the first detector, and acquire the second parameter from the second detector.

6. The controller according to claim 5, wherein the processor is configured to:
determine that the thickness of the first portion of the treatment target is greater than the thickness of the second portion of the treatment target when the first parameter is larger than the second parameter; and
determine that the thickness of the first portion of the treatment target is less than the thickness of the second portion of the treatment target when the first parameter is smaller than the second parameter.

7. The controller according to claim 5, wherein:
the first detector is configured to detect a pressure acting on the first counter electrode as the first parameter, and
the second detector is configured to detect a pressure acting on the second counter electrode as the second parameter.

8. The controller according to claim 5, wherein:
the first detector is configured to detect a displacement generated in the first counter electrode as the first parameter, and
the second detector is configured to detect a displacement generated in the second counter electrode as the second parameter.

9. The controller according to claim 1, wherein the second counter electrode is disposed at a position different from a position of the first counter electrode in a width direction of the second grasping piece.

10. The controller according to claim 1, wherein the second counter electrode is disposed at a position different from a position of the first counter electrode in a longitudinal direction of the second grasping piece.

11. The controller according to claim 1, wherein the reference electrode includes a first reference electrode facing the first counter electrode, and a second reference electrode electrically insulated from the first reference electrode and facing the second counter electrode.

12. The controller according to claim 1, further comprising:
a first detector provided on the first counter electrode and configured to detect the first parameter relating to the thickness of the first portion of the treatment target, the first portion being grasped between the reference electrode and the first counter electrode; and
a second detector provided on the second counter electrode and configured to detect the second parameter relating to the thickness of the second portion of the treatment target, the second portion being grasped between the reference electrode and the second counter electrode.

13. A treatment system comprising:
an energy treatment instrument including:
a first grasping piece,
a second grasping piece configured to grasp a treatment target by opening and closing relative to the first grasping piece,
a reference electrode provided in the first grasping piece,
a first counter electrode provided in the second grasping piece, and
a second counter electrode provided in the second grasping piece; and
a controller including a processor configured to:
acquire (i) a first parameter relating to a thickness of a first portion of the treatment target, the first portion being grasped between the reference electrode and the first counter electrode, and (ii) a second parameter relating to a thickness of a second portion of the treatment target, the second portion being grasped between the reference electrode and the second counter electrode,
calculate a difference value between the first parameter and the second parameter, and
control an amount of electric energy applied to one of the first portion or the second portion, which has a greater thickness, of the treatment target, such that the amount of electric energy applied is larger than an amount of electric energy applied to another one of the first portion or the second portion, which has a lesser thickness, of the treatment target when the calculated difference value is greater than a predetermined value.

14. A method for actuating the controller according to claim 1, the method comprising:
acquiring the first parameter relating to the thickness of the first portion of the treatment target, the first portion being grasped between the reference electrode and the first counter electrode, and the second parameter relating to the thickness of the second portion of the treatment target, the second portion being grasped between the reference electrode and the second counter electrode;
calculating the difference value between the first parameter and the second parameter; and
controlling the amount of the electric energy applied to the one of the first portion or the second portion, which has the greater thickness, of the treatment target, such that the amount of electric energy applied is larger than the amount of the electric energy applied to the other one of the first portion or the second portion, which has the lesser thickness, of the treatment target when the calculated difference value is greater than the predetermined value.

15. The method according to claim 14, wherein acquiring the first parameter and the second parameter includes calculating, as the first parameter, a first impedance value in a first electric circuit that supplies electric energy to the first counter electrode, and calculating, as the second parameter, a second impedance value in a second electric circuit that supplies electric energy to the second counter electrode.

16. The method according to claim 14, wherein:
the energy treatment instrument used together with the controller further includes:
a first detector provided on the first counter electrode and configured to detect the first parameter; and
a second detector provided on the second counter electrode and configured to detect the second parameter, and
the acquiring of the first parameter and the second parameter includes acquiring the first parameter from the first detector, and acquiring the second parameter from the second detector.
